(19) United States
(12) Patent Application Publication
(10) Pub. No.: US 2023/0107491 A1
(43) Pub. Date: Apr. 6, 2023

(54) METHOD FOR PREVENTING OR REDUCING CANNABACEAE BIOMASS DECOMPOSITION

(71) Applicant: PURCANN PHARMA INC., Québec, CA (US)

(72) Inventors: Simon GIRET, Québec (CA); André GOSSELIN, Saint-Laurent-de-l'Île-d'Orléan (CA); Christophe MELLON, Québec (CA)

(21) Appl. No.: 17/792,060

(22) PCT Filed: Jan. 29, 2021
(86) PCT No.: PCT/CA2021/050093

§ 371 Date: Jul. 11, 2022

Related U.S. Application Data
(60) Provisional application No. 62/967,648, filed on Jan. 30, 2020.

(51) Int. Cl. B65B 31/04; A01N 3/00; B65D 81/20; B65B 63/02; B65D 81/30

(52) U.S. Cl. CPC B65B 31/048; A01N 3/00; B65D 81/2084; B65B 63/02; B65D 81/30

(57) ABSTRACT

The present disclosure relates to a method for preventing or reducing cannabaceae biomass decomposition during storage by packaging an amount of the biomass in a flexible film of gas impervious material to provide a cannabaceae biomass package which is impermeable to gases.

### FIELD OF THE DISCLOSURE

The present disclosure relates to a method for preventing or reducing cannabaceae biomass decomposition during storage.

### BACKGROUND

The cannabinoids molecules have been studied for various reasons over the past decades. In recent years, the use of cannabinoids as pharmaceutical, cosmetic or recreational products has increased in several countries.

Currently, the major sources of cannabinoids are coming from the extraction of Kemps and/or Cannabis biomass. However, due to their sensitivity to temperature rise, oxidation and light, the protection of cannabinoids after harvesting and during storage is essential to preserve the economic potential of the cannabinoid biomass.

### SUMMARY OF THE DISCLOSURE

This disclosure provides a method for preventing or reducing cannabaceae biomass decomposition comprising: packaging an amount of said biomass in a flexible film of gas impervious material to provide a cannabaceae biomass package which is impermeable to gases; and exhausting the air from said cannabaceae biomass package.

A further aspect relates to a cannabaceae biomass package prepared by the method as defined herein.

A further aspect relates to a cannabaceae biomass package as defined herein.

In an embodiment, the step of exhausting the air from said cannabaceae biomass package is comprising applying at least a partial vacuum to remove said air from said package.

In a further embodiment, the step of exhausting the air from said cannabaceae biomass package comprises applying at least a partial vacuum to remove said air from said package and replacing at least a portion of the exhausted air with an inert gas.

In another embodiment, the step of exhausting the air from said cannabaceae biomass package comprises replacing at least a portion of said air with an inert gas.

In a further embodiment, the step of exhausting the air from said cannabaceae biomass package comprises exhausting at least a portion of said air from within said biomass package and simultaneously admitting an inert gas within said biomass package.

In an embodiment, the inert gas is a substantially oxygen free gas.

In a further embodiment, the inert gas is nitrogen or carbon dioxide.

In another embodiment, the method provided herein further comprises a step of sealing the cannabaceae biomass package after the step of exhausting the air.

In another embodiment, the method provided herein further comprises a step of pre-treating the cannabaceae biomass before the step of packaging.

In a supplemental embodiment, the pre-treating step comprises drying the cannabaceae biomass.

In an additional embodiment, the drying step is reducing moisture of the cannabaceae biomass to less than about 75%(wt/wt ratio).

In an embodiment, the pre-treating step comprises grinding the cannabaceae biomass.

In another embodiment, the pre-treating step comprises a step of compacting said cannabaceae biomass.

In an embodiment, preventing or reducing cannabaceae biomass decomposition comprises preventing or reducing decomposition of at least one cannabinoid compound selected from tetrahydrocannabinol (THC), tetrahydrocannabinolic acid (THCA), cannabichromene (CBC), cannabidiol (CBD), cannabidiolic acid (CBDA), cannabigerol (CBG), cannabinol (CBN), cannabinodiol (CBND), cannabicyclol (CBL), cannabielsoin (CBE), cannabicitran type (CBCT), cannabitriol (CBT), cannabielsoin (CBL) or sesquicannabigerol, cannabicoumarononic acid, cannabinoid benzoquinone, and cannabispiroindane.

In another embodiment, the preventing or reducing cannabaceae biomass decomposition comprises preventing or reducing decomposition of at least one carboxylic acid-containing cannabinoid compound, and wherein the decomposition is decarboxylation or partial decarboxylation of the carboxylic acid-containing cannabinoid compound.

In an embodiment, the carboxylic acid-containing cannabinoid compound is comprising THCA and CBDA.

In a further embodiment, the cannabaceae biomass decomposition is assessed by comparing an amount of at least one cannabinoid compound in the packaged cannabaceae biomass with reference to an initial amount of the at least one cannabinoid compound in the cannabaceae biomass measured before the step of packaging.

In another embodiment, the cannabaceae biomass is at least one of hemp, Cannabis sativa, Cannabis indica, a hybrid of Cannabis sativa and indica, Cannabis ruderalis, Humulus (hops), and Celtis (hackberries).

In an embodiment, the flexible film of gas impervious material protects against air, moisture penetration, U.V. treated to resist ultra violet light degradation, with high tear and puncture resistance.

In another embodiment, the flexible film of gas impervious material is multilayered film or a laminated film.

In a supplemental embodiment, the flexible film of gas impervious material is silage wrap film or silage stretch tube.

In a further embodiment the flexible film is made of low-density polyethylene.

### BRIEF DESCRIPTION OF THE FIGURES

FIG. 1. is a graph representing the storage room temperature and the packaged cannabaceae biomass temperature changes in relation to the elapsed time.

### DETAILED DESCRIPTION

The present disclosure relates to a method for preventing or reducing cannabaceae biomass decomposition during storage.

It is believed that the packaging method described herein is useful to prevent or reduce decomposition and/or alteration in the chemical structure of certain cannabinoids. It is thus provided a packaging method that limits the aerobic digestion, the temperature rise and further avoids air oxidation of the cannabaceae biomass.

In one embodiment, the cannabaceae biomass is at least one of hemp, Cannabis sativa, Cannabis indica, a hybrid of Cannabis sativa and indica, Cannabis ruderalis, Humulus (hops), and Celtis (hackberries). The Cannabaceae biomass may be obtained from any part of cannabaceae plant.

Various flexible films of gas impervious material may be employed in packaging said biomass in accordance with the process described herein however the film which is employed should be impermeable to carbon dioxide and oxygen. In accordance with the disclosure, films such as multilayered, or laminated films may be used. In an embodiment, the films are silage wrap film or silage stretch tubes, or any film effective for the fermentation and storage, preferably that protects against air (oxygen free environment) and moisture penetration, U.V. treated to resist ultra violet light degradation for up to one year, and with high tear and puncture resistance. In a particular embodiment, the film is made of low-density polyethylene.

There are a number of commercially available, flexible films made of gas impervious material on the market which are, satisfactory for use in this method.

The flexible film of gas impervious material may also preferably be opaque to light and UV, thereby reducing the cannabaceae biomass decomposition. Alternatively, a less opaque or transparent biomass package may be stored in a shaded or dark area/storage facility.

At a larger scale, it might be desired to package the cannabaceae biomass using a silage-like process such as by piling the biomass in a large heap and compressing it down so as to purge as much oxygen as possible, and then wrapping the cannabaceae biomass with the flexible film of gas impervious material in large round or cube bale.

In one embodiment, before the step of packaging, the method comprises a further step of pre-treating the cannabaceae biomass.

In one embodiment, said pre-treating step comprises drying the cannabaceae biomass.

In one embodiment, said pre-treating step comprises grinding the cannabaceae biomass.

The grinding step is intended to cause a size reduction of the biomass. Examples include the shredding of the cannabaceae biomass to pieces about 1 to 0.1-inch-long, preferably about 0.5 inch long.

In one embodiment, said pre-treating step comprises grinding and drying the cannabaceae biomass.

In one embodiment the drying step reduces moisture of the cannabaceae biomass to less than about 75%(wt/wt ratio) or below about 50%.

In one embodiment, the cannabaceae biomass moisture content to be packaged is between about 35% to about 60%, more preferably of at least about 35%, or at least about 40%, or at least about 50% or at least about 60%. Accordingly, the pre-treating step comprising drying the cannabaceae biomass allows to reduce the initial humidity of the biomass.

In one embodiment, the drying is conducted at room temperature (i.e. from about 20-30° C.).

In one embodiment, the drying is conducted at a temperature below 100° C.

In one embodiment, said pre-treating step comprises a step of compacting said cannabaceae biomass. The compacting step may be conducted by mechanical compression. The compression should be applied with enough force to avoid the diffusion of outside air inside the biomass during the conservation. The compacting step may be conducted on cannabaceae biomass obtained after said grinding and/or drying of the biomass described herein. Ideal silage compaction on a dry matter basis should reach a density of 15 lbs/cubic foot or 240 kg/cubic metre of dry matter.

In one embodiment, said exhausting the air from said cannabaceae biomass package comprises applying at least a partial vacuum to remove at least a portion of said air from said package.

In one embodiment, said exhausting the air from said cannabaceae biomass package comprises applying at least a partial vacuum to remove said air from said package.

In one embodiment, said exhausting the air from said cannabaceae biomass package comprises applying at least a partial vacuum to remove at least a portion of said air from said package and back-filling all or at least a portion of the exhausted air with an inert gas.

In one embodiment, said exhausting the air from said cannabaceae biomass package comprises applying at least a partial vacuum to remove air from said package and back-filling at least a portion of the exhausted air with an inert gas.

In one embodiment, said method further comprises replacing the exhausted air with an inert gas.

In one embodiment, said exhausting the air from said cannabaceae biomass package comprises exhausting said air from within said biomass package and simultaneously admitting said inert gas within said biomass package.

In one embodiment, said inert gas is a substantially oxygen free gas, such as nitrogen or carbon dioxide.

In one embodiment, said method further comprises sealing said package.

In one embodiment, said cannabaceae biomass decomposition is assessed by the temperature rise (preferably the average measured temperature) of the packaged biomass relative to the temperature of the facility or room where the biomass package is stored. Preferably the temperature of the storage facility or room ranges from about above 0° C. to about 50° C., or from about 5° C. to about 40° C., or from about 10° C. to about 30° C., or from about 20° C. to about 25° C. The temperature of the cannabaceae biomass may conveniently be monitored on the outside of the package, as a convenient way to measure the temperature of the packaged biomass, for example by using infra-red thermometer. This method is convenient, although it is understood that the temperature inside the biomass may be slightly higher, as it avoids puncturing the packaged biomass.

In one embodiment, the packaged biomass temperature rise, relative to a reference storage temperature during the storage time, is less than about 40° C., or less than about 20° C., or less than about 10° C. or less than about 2-5° C., alternatively the packaged biomass temperature rise is comprised between about 5 and 40° C.

In one embodiment, said cannabaceae biomass decomposition is assessed by comparing an amount of at least one cannabinoid compound in said packaged cannabaceae biomass with reference to an initial amount of said at least one cannabinoid compound in said cannabaceae biomass measured before said step of packaging.

In one embodiment, the cannabinoid compound is at least one of tetrahydrocannabinol (THC) (such as Δ9-THC, Δ8-THC), cannabichromene (CBC), cannabidiol (CBD), cannabigerol (CBG), cannabinol (CBN), cannabinodiol (CBND), cannabicyclol (CBL), cannabielsoin (CBE), cannabicitran type (CBCT), cannabitriol (CBT), cannabielsoin (CBL) or other types (such sesquicannabigerol; cannabicoumarononic acid; cannabinoid benzoquinone; cannabispiroindane).

In one embodiment, said at least one cannabinoid compound is one or more of THC, THCA, CBD and CBDA.

The relevant compound structures are summarized in the following table:

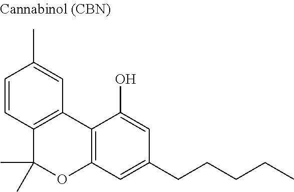 [SMILES: CCCCCc1cc(O)c2c(c1)OC(C)(C)c1ccc(C)cc1-2]

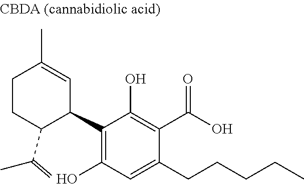 [SMILES: C=C(C)[C@@H]1CCC(C)=C[C@H]1c1c(O)cc(CCCCC)c(C(=O)O)c1O]

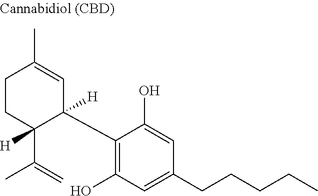 [SMILES: C=C(C)[C@@H]1CCC(C)=C[C@H]1c1c(O)cc(CCCCC)cc1O]

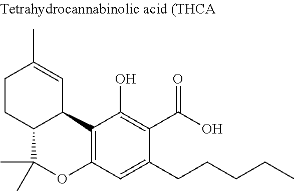 [SMILES: CCCCCc1cc2c(c(O)c1C(=O)O)[C@@H]1C=C(C)CC[C@H]1C(C)(C)O2]

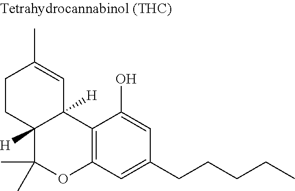 [SMILES: CCCCCc1cc(O)c2c(c1)OC(C)(C)[C@@H]1CCC(C)=C[C@@H]21]

In one embodiment, said cannabaceae biomass decomposition is assessed by comparing an amount of one or more of THC, THCA, CBD and CBDA in said packaged cannabaceae biomass with reference to an initial amount of those in said cannabaceae biomass measured before said step of packaging. A relative decrease in one or more of THC, THCA, CBD and CBDA or alternatively in the combined amount of said THC, THCA and/or the combined amount of said CBD and CBDA is indicative of cannabaceae biomass decomposition.

The respective decarboxylation of THCA and CBDA into THC and CBD is a known alteration process of cannabinoid in cannabaceae biomass.

In one embodiment, the method herein allows for reducing decarboxylation (e.g. only a partial decarboxylation) of carboxylic acid containing cannabinoid compounds.

In one embodiment, the partial decarboxylation of the cannabinoid compounds is less than about 50% (molar ratio), preferably less than about 33% (molecular ratio) or is preferably not detected when measured by the method described herein (e.g. HPLC method described in the examples).

In one embodiment, said at least one cannabinoid compound comprises CBN.

In one embodiment, said cannabaceae biomass decomposition is assessed by comparing an amount of CBN in said packaged cannabaceae biomass with reference to an initial amount of said CBN in said cannabaceae biomass measured before said step of packaging. A relative increase in the amount of said CBN is indicative of cannabaceae biomass decomposition.

In one embodiment, said biomass decomposition is an oxidative decomposition of at least one cannabinoid compound.

It is known in the art that CBN is synthesized by certain varieties of cannabaceae plants by biosynthesis, however it is also considered as an oxidative degradation product of certain other cannabinoid compounds, for example in accordance with the following scheme with reference to degradation of THC:

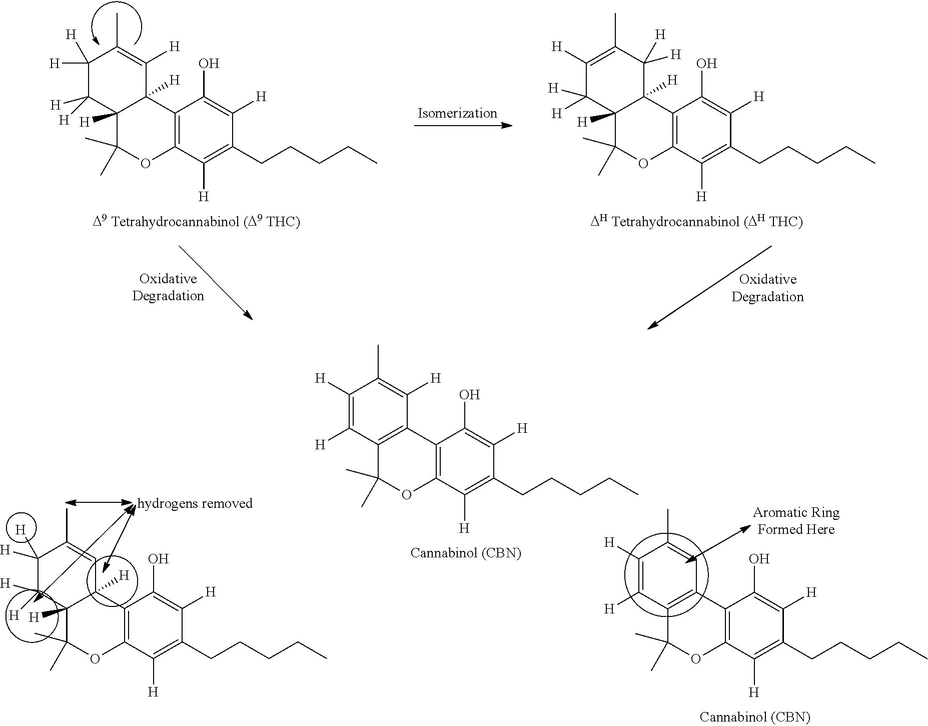

### EXAMPLE 1

### Packaging of Cannabis leaves.

During the flowering stage of Cannabis plants, cultivar Kritical Kush, Cannabis leaves were collected and stored at −15° C. After harvesting, the leaves were manually homogenized, then separated in 10 bags (Foodsaver™, transparents bag) of approximately 100 g each. The undried homogenized leaves were then compacted and sealed under vacuum with a FoodSaver® Countertop Vacuum Sealing System (see Table 1).

TABLE 1
Mass of leaves in bags.

| | Bags No | | | | | | | | | |
|---|---|---|---|---|---|---|---|---|---|---|
| | 1 | 2 | 3 | 4 | 5 | 6 | 7 | 8 | 9 | 10 |
| Mass (g) | 104.59 | 104.83 | 104.54 | 105.23 | 104.99 | 105.23 | 105.16 | 105.23 | 105.46 | 105.26 |

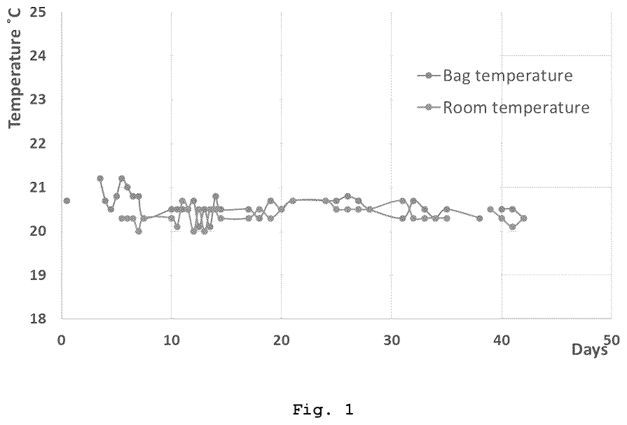

The temperature of the bags was monitored every day using an infra-red thermometer (model 057-4632-8 from Maximum®). The corresponding data are reported in FIG. 1. The temperature change was not more than about 1° C. compared to the room temperature (i.e. the temperature of the cabinet where the bags were stored). The temperature inside the packaged biomass may be slightly different (higher).

The cannabinoid composition of the packaged biomass of the 10 bags were analysed by HPLC. The analytical method was based on De Backer, B.; et al Innovative Development and Validation of an HPLC/DAD Method for the Qualitative and Quantitative Determination of Major Cannabinoids in Cannabis Plant Material. J. chromatogr. B (2009), 877, pp 4115-4124. The HPLC used was an Agilent 1200, with a DAD 1100.

All chromatographic runs were carried out using Agilent 1100 HPLC Series, consisting of a G1322A solvent degasser, a G1311A quaternary solvent pump, a G1313A autosampler, a G1316A column compartment and a G1315B photodiode-array detector. For this study the detector was set to 230 nm. Chromatographic separations were performed using a SiliaChrom dt C18 3 μm 4.6×150 mm. Column temperature was set to 35° C. and the flow was set to 1.5 mL/min. The analytical method was based on De Backer, B.; et al Innovative Development and Validation of an HPLC/DAD Method for the Qualitative and Quantitative Determination of Major Cannabinoids in Cannabis Plant Material. J. chromatogr. B (2009), 877, pp 4115-4124. In summary, samples were prepared by dilution in 100% HPLC grade MeOH, to a concentration expected to be near 50 ppm (i.e. center of the standard calibration curve). The injection volume was 10 μL and the sample was eluted under isocratic condition with 75/25 (v/v) acetonitrile/ammonium formate 50 mM+0.1% (v/v) formic acid.

The results are summarized in Table 2.

TABLE 2
Assessment of cannabinoids content storage.

| | Time | THC | THCA | CBD | CBDA | CBN | THC + |
|---|---|---|---|---|---|---|---|
| | (Days) | (%) | (%) | (%) | (%) | (%) | THCA |
| flowering | 0 | 0.06 | 1.78 | 0.00 | 0.00 | 0.00 | 1.84 |
| leaves | 15 | 0.96 | 0.88 | 0.00 | 0.00 | 0.00 | 1.84 |
| | 30 | 1.14 | 0.77 | 0.00 | 0.00 | 0.00 | 1.91 |
| | 45 | 1.24 | 0.72 | 0.00 | 0.00 | 0.00 | 1.96 |

After 45 days, no CBN was detected, therefore suggesting that no significant oxidative degradation occurred. The decarboxylation reaction (i.e. conversion of THCA to THC) took place mainly during the first 15 days where more than 50% of the THCA was decarboxyled in THC. The decarboxylation continued slowly after the first 15 days to reach almost 66% after 45 days.

While the present disclosure has been described in connection with specific embodiments thereof, it will be understood that it is capable of further modifications and this application is intended to cover any variations, uses, or adaptations including such departures from the present disclosure as come within known or customary practice within the art and as may be applied to the essential features hereinbefore set forth, and as follows in the scope of the appended claims.

1. A method for preventing or reducing cannabaceae biomass decomposition comprising:
packaging an amount of said biomass in a flexible film of gas impervious material to provide a cannabaceae biomass package which is impermeable to gases; and
exhausting the air from said cannabaceae biomass package.

2. The method according to claim 1, wherein said step of exhausting the air from said cannabaceae biomass package is comprising applying at least a partial vacuum to remove air from said cannabaceae biomass package.

3. The method according to claim 1, wherein said step of exhausting the air from said cannabaceae biomass package comprises applying at least a partial vacuum to remove air from said cannabaceae biomass package and replacing at least a portion of the exhausted air with an inert gas.

4. The method according to claim 1, wherein said step of exhausting the air from said cannabaceae biomass package comprises replacing at least a portion of air with an inert gas.

5. The method according to claim 1, wherein said step of exhausting the air from said cannabaceae biomass package comprises exhausting at least a portion of air from within said cannabaceae biomass package and simultaneously admitting an inert gas within said cannabaceae biomass package.

6. The method according to claim 3, wherein said inert gas is a substantially oxygen free gas.

7. The method according to claim 6, wherein said inert gas is nitrogen or carbon dioxide.

8. The method according to claim 1, further comprising a step of sealing said cannabaceae biomass package after said step of exhausting the air.

9. The method according to claim 1, further comprising a step of pre-treating the cannabaceae biomass before the step of packaging.

10. The method according to claim 1, wherein said pre-treating step comprises drying the cannabaceae biomass.

11. The method according to claim 10, wherein said drying step is reducing moisture of the cannabaceae biomass to less than about 75% (wt/wt ratio).

12. The method according to claim 9, wherein said pre-treating step comprises grinding the cannabaceae biomass.

13. The method according to claim 9, wherein said pre-treating step comprises a step of compacting said cannabaceae biomass.

14. The method according to claim 1, wherein preventing or reducing cannabaceae biomass decomposition comprises preventing or reducing decomposition of at least one cannabinoid compound selected from tetrahydrocannabinol (THC), tetrahydrocannabinolic acid (THCA), cannabichromene (CBC), cannabidiol (CBD), cannabidiolic acid (CBDA), cannabigerol (CBG), cannabinol (CBN), cannabinodiol (CBND), cannabicyclol (CBL), cannabielsoin (CBE), cannabicitran type (CBCT), cannabitriol (CBT), cannabielsoin (CBL) or sesquicannabigerol, cannabicoumarononic acid, cannabinoid benzoquinone, and cannabispiroindane.

15. The method according to claim 1, wherein said preventing or reducing cannabaceae biomass decomposition comprises preventing or reducing decomposition of at least one carboxylic acid-containing cannabinoid compound, and wherein said decomposition is decarboxylation or partial decarboxylation of said carboxylic acid-containing cannabinoid compound.

16. The method according to claim 15, wherein said carboxylic acid-containing cannabinoid compound is comprising THCA and CBDA.

17. The method according to claim 1, wherein said cannabaceae biomass decomposition is assessed by comparing an amount of at least one cannabinoid compound in said packaged cannabaceae biomass with reference to an initial amount of said at least one cannabinoid compound in said cannabaceae biomass measured before said step of packaging.

18. The method according to claim 1, wherein said cannabaceae biomass is at least one of hemp, Cannabis sativa, Cannabis indica, a hybrid of Cannabis sativa and indica, Cannabis ruderalis, Humulus (hops), and Celtis (hackberries).

19. The method according to claim 1, wherein said flexible film of gas impervious material protects against air, moisture penetration, U.V. treated to resist ultra violet light degradation, with high tear and puncture resistance.

20-22 (canceled)

23. A cannabaceae biomass package prepared by the method as defined in claim 1.

24. (canceled)
